(19) United States
(12) Patent Application Publication
(10) Pub. No.: US 2004/0122414 A9
(43) Pub. Date: Jun. 24, 2004

(54) Contoured tubing closure

(76) Inventors: William S. Hurst, Burlington, WI (US); Brian Adamkiewicz, Cary, IL (US); Hermann F. Eckert, Palatine, IL (US); Janelle Herren, Medford, MA (US); Sidney T. Smith, Lake Forest, IL (US)

Correspondence Address: BAXTER HEALTHCARE CORPORATION, RENAL DIVISION, 1 BAXTER PARKWAY, DF3-3E, DEERFIELD, IL, 60015, US

(21) Appl. No.: 10/097,200

(22) Filed: Mar. 12, 2002

Related U.S. Application Data
(63) Continuation of application No. 09/607,058, filed on Jun. 29, 2000, now Pat. No. 6,361,843.
Application No. 09/607,058 is a continuation of application No. 08/934,924, filed on Sep. 22, 1997, now Pat. No. 6,083,587.

(51) Int. Cl. A61M 5/32

(52) U.S. Cl. 604/415; 264/346

(57) ABSTRACT

The present invention provides a system and method for delivering fluid. In one embodiment, the system and method include a container to hold the fluid, and a closed-end tube having a first end in communication with the container and a closed second end. The closed second end is contoured in a pattern to form a zone of weakness. The zone of weakness facilitates reduced force necessary for an access spike to puncture the closed second end.

### BACKGROUND OF THE INVENTION

The present invention relates to a system and method for delivering fluids. In particular, researchers are continually developing medical fluids to treat patients for a wide range of medical conditions. Such fluids can include intravenous solutions, nutritional solutions, drug solutions, blood, blood components, blood substitutes such as deoxygenated hemoglobins, dialysate fluids, cell culture media, bioprocessing fluids containers for therapeutic products such as Factor VIII, or other fluids that might be delivered to a patient.

Medical and other fluid delivery systems typically include a container that holds the fluid, and tubing in communication with the container that delivers the fluid. The container is often a polymeric film bag or pouch designed to hold the particular fluid. The container may also be a glass bottle, or any other container suitable for holding the fluid.

Numerous polymeric films have been developed for use in containers. Container films may be a monolayer structure or a multiple layer structure of polymeric materials formed as a pouch or bag. The monolayer structure can be made from a single polymer, or from a polymer blend. Multiple layer structures can be formed by co-extrusion, extrusion lamination, lamination, or any suitable means. The multiple layer structures can include layers such as a solution contact layer, a scratch resistant layer, a barrier layer for preventing ingress of oxygen or water vapor, tie layers, or other layers.

The pouch can be formed by placing two polymeric film sheets in registration by their peripheral portions and sealing the outer periphery to form a fluid tight pouch. The sheets are typically sealed by heat sealing, radio frequency sealing, thermal transfer welding, adhesive sealing, solvent bonding, and ultrasonic or laser welding.

Blow molding is another method used to make the pouch. Blow molding is a blown extrusion process that provides a moving tube of extrudate exiting an extrusion die. Air under pressure inflates the tube. Longitudinal ends of the tube are sealed to form the pouch. Blow molding only requires seals along two peripheral surfaces, where the registration method requires seals along four peripheral surfaces to form the pouch.

Medical fluid containers commonly provide ports for access to medical fluid contained within them. For pouch or bag containers, access ports typically are a tube inserted between the sidewalls of the container, or attached to a sidewall of the container. A membrane tube is typically inserted into the access port tube. The membrane tube is often solvent bonded to the access port tube. In solvent bonding, the membrane tube is dipped into solvent, and then inserted into the port tube. Thus, the outer surface of the membrane tube becomes bonded to the inner surface of the access port tube.

The membrane tube defines a passageway which permits fluid communication between the container and tubing which delivers the medical fluid to the patient. A membrane is typically disposed across the passageway to seal the medical fluid in the container until the fluid is to be delivered. The membrane also helps to preserve fluid that may be sensitive to the atmosphere. For example, the fluid may degrade in the presence of oxygen. To access fluid in the container, a hollow access spike is typically inserted into the access port. When inserted sufficiently into the access port, the access spike punctures the membrane thereby allowing fluid to flow from the container.

Conventional solution containers employing access ports typically use access port materials of flexible polyvinyl chloride (PVC) or soft polyolefins such as low density polyethylene (LDPE). These materials have sufficient elasticity to grip the outside of the access spike to retain the spike during fluid delivery. The inner diameter of the end port is dimensioned slightly smaller than the outer diameter of the access spike. The elasticity of PVC or LDPE is sufficient to permit the end port to expand about the outside of the access spike forming an interference fit.

Researches are also continually developing medical and other therapeutic or nutritional solutions that have unusual and specific container requirements. These requirements can include providing a gas barrier to prevent contamination or degradation of the medical fluid within the container by contact with gases. For example, ethylene (vinyl alcohol) (EVOH) provides a high barrier to the ingress of oxygen. EVOH may be used as a barrier layer in a laminate of polymeric material or co-extruded with the polymeric material. The membrane which seals the container is often also made of a polymeric material combined with a barrier material layer such as EVOH. The inclusion of EVOH, however, in a film increases the film's rigidity. This may make the membrane containing EVOH difficult to puncture with the typical access spike.

Moreover, some solutions require containers having increased reactive inertness with respect to the solution. For example, proteins, blood, blood components and biologically active substances can be denatured by contact with the polymer molecules of the container. Polymeric materials with increased inertness used to manufacture containers or membranes also typically have a higher modulus or elasticity, and are more difficult to puncture with an access spike than containers not requiring additional inertness.

In the interest of safety, fluid delivery systems are also trending away from needles, to needleless systems. Needleless systems include blunt cannulas in increasing use in the medical field. Needleless systems eliminate, or at least lessen, the chance of a medical worker accidentally incurring a needle stick. Needleless systems, therefore, protect the medical worker from accidental exposure to blood-borne pathogens. They also help prevent contamination of the medical fluid. The trend to needleless systems, combined with the use of increasingly rigid materials in medical fluid packaging make the seals of the container difficult to puncture using typical access spikes. Difficulty in puncturing may result in the container, access port, membrane tube, or membrane being torn. It may also cause a break of the interference fit between the access port and the access spike. These conditions may cause the medical fluid to leak from the container. It may also result in contamination or degradation of the medical fluid because of contact with the atmosphere.

For renal fluid applications, the delivery to the patient typically requires multiple fluids be delivered to the patent in succession. These fluids may consist of two or more different fluids that must be delivered to the patient during a treatment session, or two or more containers of the same fluid, or switching from one fluid to another, and back. Thus, for renal applications, a disconnectable and reconnectable fluid delivery system that prevents leakage from a renal fluid container is desirable. Moroever, frangible tubes used in renal systems must be snapped and then wiggled to remove the frangible.

The present invention provides a medical delivery system and method that addresses these and other problems.

### SUMMARY OF THE INVENTION

The present invention provides a system and method for delivering fluid. In one embodiment, the system and method include a container to hold the fluid, and a closed-end tube having a first end in communication with the container and a closed second end. The closed second end is contoured in a pattern to form a zone of weakness. The zone of weakness facilitates reduced spike force access, i.e., the force necessary for an access spike to puncture the closed second end.

In another embodiment of the present invention, the system and method include a container for holding the fluid, a passageway in communication with the container, and a membrane disposed across the passageway to seal the passageway. The membrane is contoured in a pattern to define a zone of weakness. The zone of weakness again provides the advantage of reduced spike access force.

In a further embodiment of the present invention, the system and method include a container for holding the fluid, and a tube defining a passageway in communication with the container. The tube has a membrane disposed across the passageway, and is contoured in a pattern to define a zone of weakness. There is an interface in the tube between the membrane and an end of the tube, and a connector inserted into the end of the tube. The connector is adapted to engage the interface, and to cause the interface to puncture the membrane thereby delivering the fluid through the passageway. The present invention, therefore, permits a low access force for use with in-line frangibles for renal applications.

A further embodiment of the present invention includes a capsule having a body, the body having a first end and a second end, and at least one of the first end or second end contoured to define a zone of weakness. A still further embodiment includes a fluid mixing system having a capsule, the capsule having a first end and a second end, and at least one of the first end or second end contoured to define a zone of weakness, the capsule contained within a container, the capsule containing a first material, and the container containing a second material.

In another aspect of the present invention, the contouring also permits resealing of the membrane after puncturing. Additional features and advantages of the present invention are described in, and will be apparent from, the following Detailed Description of the Invention and the figures.

### BRIEF DESCRIPTION OF THE FIGURES

FIG. 1 is a cross-sectional view of a molded closed end tube in accord with the present invention.

FIG. 2 is a cross-sectional view of a series of extrusion molded closed end tubes in accord with the present invention.

FIG. 3 is a cross-sectional view of an extruded tube and a sealed tube in accord with the present invention.

FIG. 4 is a contouring pattern in accord with one embodiment of the present invention.

FIG. 5 is a contouring pattern in accord with another embodiment of the present invention.

FIG. 6 is a contouring pattern in accord with a further embodiment of the present invention.

FIG. 7A is side view of a contouring pattern in accord with an additional embodiment of the present invention.

FIG. 7B is an end view of the contouring pattern of FIG. 7A.

FIG. 8A shows one method of contouring in accord with one embodiment of the present invention.

FIG. 8B shows a later step of the contouring method of FIG. 8A.

FIG. 8C shows a later step of the contouring method of FIG. 8B.

FIG. 9A is a cross-sectional view of an embodiment of the present invention.

FIG. 9B is a cross-sectional view of the embodiment of FIG. 9A.

FIG. 9C is a cross-sectional view of the embodiment of FIG. 9A.

FIG. 10 is a cross-sectional view of another embodiment of the present invention.

FIG. 11 is a cross-sectional view of a further embodiment of the present invention.

FIG. 12 is a plan view of a center access spike.

FIG. 13 is a plan view of a bevel access spike.

FIG. 14 is a plan view of a typical medical fluid delivery system.

FIG. 15 is a cross-sectional view of a three-layered tubing.

FIG. 16 is a cross-sectional view of a two-layered tubing.

FIG. 17 is a cross-sectional view of a two-layered membrane film.

FIG. 18 is a cross-sectional view of a three-layered membrane film.

FIG. 19 is a cross-sectional view of a five-layered membrane film.

FIG. 20 is a side view of a spike holder.

FIG. 21 is an end view taken along line A-A of FIG. 20.

FIG. 22 is cross-sectional view of a spike holder assembly.

FIG. 23 is a cross-section area of one embodiment of a membrane in accord with the present invention.

FIG. 24 is a cross-sectional view of a further embodiment of the present invention.

FIG. 25 is a plan view of a capsule embodiment of the present invention

FIG. 26 is a plan view of an embodiment incorporated the capsule of FIG. 25.

### DETAILED DESCRIPTION OF THE INVENTION

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims. For example, though described below with respect to a medical fluid delivery application, this invention may be used in other fluid delivery applications such as food or chemical industry packaging and delivery.

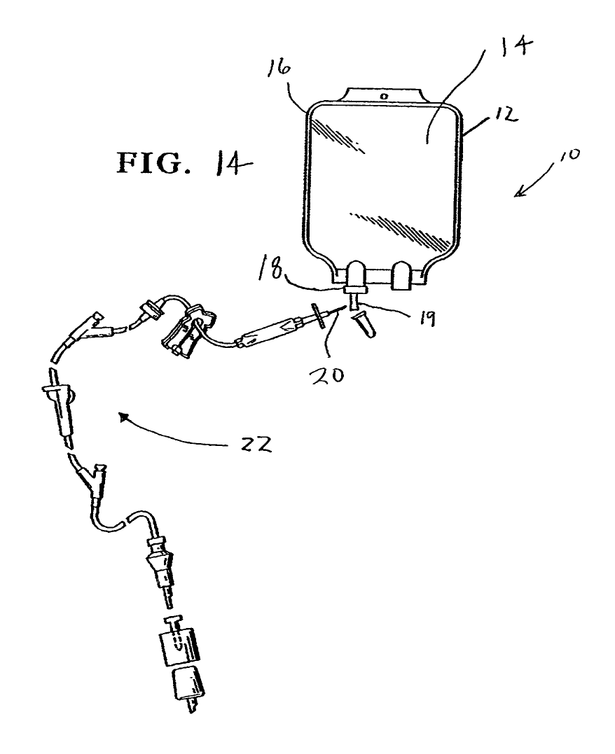

FIG. 14 generally illustrates an intravenous medical delivery system 10 used in one embodiment of the present invention. FIG. 14 shows a container 12 for holding medical fluids 14 for delivery to a patient (not shown). The medical fluids 14 may include intravenous solutions, nutritional solutions, drug solutions, blood, blood components, blood substitutes such as deoxygenated hemoglobins, renal fluids, cell culture, recombinant DNA fluids for forming therapeutic products such as Factor VIII, or other fluids that have a therapeutic effect.

The container 12 may be made of any suitable material, but is typically made of polymeric film materials. Container 12 films may be a monolayer structure or a multiple layer structure of polymeric materials formed as a pouch or bag. The monolayer structure can be made from a single polymer, or from a polymer blend. The monolayer film can be formed by extrusion or other polymer processing techniques well-known to those skilled in the art. Multiple layer films can be formed by co-extrusion, extrusion lamination, lamination, or any suitable means. The multiple layer structure can include the monolayer structure with additional layers. The additional layers can include layers such as a solution contact layer, a scratch resistant layer, a barrier layer for preventing ingress or egress of oxygen, carbon dioxide, or water vapor, tie layers or other layers.

The container 12 can be formed by placing two polymeric film sheets in registration by their peripheral portions and sealing the outer periphery 16 to form a fluid tight pouch. The sheets are sealed along their periphery 16 by heat sealing, radio frequency sealing, thermal transfer welding, adhesive sealing, solvent bonding, and ultrasonic or laser welding.

Blow molding is another method that may be used to make the container 12. Blow molding is a blown extrusion process that provides a moving tube of extrudate exiting an extrusion die. Air under pressure is used to inflate the tube. Longitudinal ends of the tube are sealed to form the pouch. Blow molding only requires seals along two peripheral surfaces, where the registration method requires seals along four peripheral surfaces 16 to form the pouch.

Films typically used to make the container 12 include layers of polymeric materials selected from the following: high density polyethylene (HDPE), medium density polyethylene (MDPE), low density polyethylene (LDPE), very low density polyethylene (VLDPE), ultra low density polyethylene (ULDPE), linear low density polyethylene (LLDPE), polypropylene, polyolefins, modified polyolefins, polyvinyl chloride (PVC), nylon, ethylene vinyl acetate (EVA), polyester, polyamides, or any other suitable material. The particular polymeric material selected will depend on the application.

For medical and other applications, it is also often desirable that films used to make container 12 include one or more layers of a barrier material. Barrier materials minimize the infiltration of gases such as oxygen, carbon dioxide, or water vapor, into the fluid 14 in the container 12. Such gases may contaminate or degrade the fluid 14, thereby decreasing or negating its usefulness. Typical barrier materials include ethylene (vinyl alcohol) (EVOH), which provides a high barrier to oxygen. Other barrier materials include polyvinylidene chloride (PVDC) and metal foils such as aluminum foil. Barrier materials may be laminated, blow molded, or co-extruded with other polymeric materials as described above. The barrier layers typically include EVOH with about 25% to about 45% ethylene content by mole percent.

For medical applications where the containers 12 are disposed by incineration, it is also desirable to construct the container 12 and other components of the fluid delivery system 10 from non-halogen containing polymers. Halogen containing compounds potentially create inorganic acids upon incineration. For medical applications, it is also desirable to construct the delivery system 10 from as small amount as possible of low molecular weight additives. Low molecular weight components, such as plasticizers can potentially leach into the fluids contained in the container 12, or transported through the delivery system 10.

The container 12 typically provides at least one access port 18 that permits access to the medical fluid 14 in the container 12. The access port 18 is generally a tube. The access port 18 is typically inserted between the container sidewalls, and is in communication with the inside of the container 12. A membrane tube 19 is inserted into the access port 18. The outer surface of the membrane tube 19 is preferably solvent bonded to the interior surface of the access port 18. The membrane tube 19 is generally sealed with a membrane (not shown) disposed across the membrane tube 19 that seals the medical fluid 14 in the container 12. To access the fluid 14 from the container 12, an access spike 20 is inserted into the membrane tube 19. When inserted, the access spike 20 punctures the membrane. Tubing 22 attached to the access spike 20 delivers the medical fluid 14 to the patient.

Typical access spikes 20 include a beveled spike 24 (FIG. 13), and center point spike 25 (FIG. 12). When a barrier material such as EVOH is used in the membrane of the fluid delivery system, it increases the force necessary to puncture the membrane using typical access spikes 24 or 25 because of EVOH's rigidity.

In another embodiment, the container 12 may be rigid and may be pressurized or evacuated. Thus, when the access spike 20 is inserted, an audible indication of puncture is heard caused by the movement of air.

In one preferred embodiment, the present invention includes a closed end membrane tube 26 (FIG. 1). Like the typical membrane tube 19, the closed end tube 26 is attached to the container 12 using any suitable process, but may be inserted between container sidewalls. The closed end tube 26 has a sidewall 28 substantially cylindrical in cross-section that defines a passageway 30. The closed end tube 26 and passageway 30, however, need not be cylindrical, but may be any suitable cross-sectional shape such as elliptical or polygonal. The passageway 30 communicates with the interior of the container 12 to permit fluid 14 to flow through the passageway 30. The closed end tube 26 has a first end 32 and a second end 34. The closed end tube 26 is closed at the second end 34. The closed second end 34 has an inner surface 35 and an outer surface 37.

While it is contemplated the closed end tube 26 can have any number of layers, in a preferred form of the invention the closed end tube 26 will include either a discrete layer of a barrier material or a blend layer including a barrier material. The barrier material will present a barrier to the passage of gases or water vapor transmission, and, in a preferred form of the invention, will reduce the passage rate of oxygen therethrough. It is also desirable that all materials in the solution contact layer, and more preferably all materials used in the tubing, be free of halogens, plasticizers or other low-molecular weight or water soluble components that can leach out into the solutions transferred through the tubing. Suitable barrier materials include ethylene (vinyl alcohol) copolymers having an ethylene content of from about 25% to about 45% by mole percent, more preferably from about 28% to about 36% by mole percent and most preferably from about 30% to about 34% by mole percent.

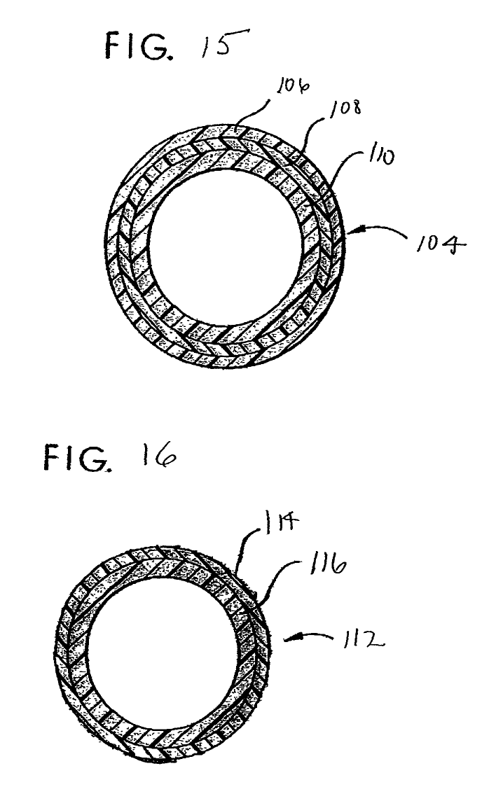

In an even more preferred form of the invention the closed end tube 26 will have multiple layers. FIG. 15 and FIG. 16 show respectively a three-layered membrane tube and a two-layered membrane tube. The three-layered membrane tube 104 has an outside layer 106, a core layer 108 and an inside solution contact layer 110. Similarly, the two-layered port tube 112 has an outside layer 114 and an inside, solution contact layer 116. The closed end 34 of the closed end tube 26 is preferably similarly constructed.

In a preferred form of the invention, the multiple layered tubings 104 and 112 will have a discrete layer of a barrier material with the remaining layers being selected from polyolefins. The layers of the tubing can be positioned in any order, however, in a preferred form of the invention the barrier layer is not positioned as the outside layer 106 or 114. Thus, the layers of a three layered tubing can be positioned in one of six orders selected from the group: first/second/third, first/third/second, second/first/third, second/third/first, third/first/second, and third/second/first. Further, in tubing embodiments having more than two layers, the tubing 104 can be symmetrical or asymmetrical from a material aspect and from a thickness of layers aspect.

Suitable polyolefins include homopolymers, copolymers and terpolymers obtained using, at least in part, monomers selected from α-olefins having from 2 to 20 carbons. One particularly suitable polyolefin is an ethylene and α-olefin interpolymer (which sometimes shall be referred to as a copolymer). Suitable ethylene and α-olefin interpolymers preferably have a density, as measured by ASTM D-792 of less than about 0.915 g/cc and are commonly referred to as very low density polyethylene (VLDPE), ultra low density ethylene (ULDPE) and the like. The α-olefin should have from 3-17 carbons, more preferably from 4-12 and most preferably 4-8 carbons. In a preferred form of the invention, the ethylene and α-olefin copolymers are obtained using single site catalysts. Suitable single site catalyst systems, among others, are those disclosed in U.S. Pat. Nos. 5,783,638 and 5,272,236. Suitable ethylene and α-olefin copolymers include those sold by Dow Chemical Company under the AFFINITY tradename, Dupont-Dow under the ENGAGE tradename and Exxon under the EXACT and PLASTOMER tradenames.

The polyolefins also include modified polyolefins and modified olefins blended with unmodified olefins. Suitable modified polyolefins are typically polyethylene or polyethylene copolymers. The polyethylenes can be ULDPE, low density (LDPE), linear low density (LLDPE), medium density polyethylene (MDPE), and high density polyethylenes (HDPE). The modified polyethylenes may have a density from 0.850-0.95 g/cc. The polyethylene may be modified by grafting or otherwise chemically, electronically or physically associating a group of carboxylic acids, and carboxylic acid anhydrides. Suitable modifying groups include, for example, maleic acid, fumaric acid, itaconic acid, citraconic acid, allylsuccinic acid, cyclohex-4-ene-1,2-dicarboxylic acid, 4-methylcyclohex-4-ene-1,2-dicarboxylic acid, bicyclo[2.2.1]hept-5-ene-2,3 -dicarboxylic acid, x-methylbicyclo[2.2.1]hept-5-ene-2,3-dicarboxylic acid, maleic anhydride, itaconic anhydride, citraconic anhyride, allylsuccinic anhydride, citraconic anhydride, allylsuccinic anhydride, cyclohex-4-ene-1,2-dicarboxylic anhydride, 4-methylcyclohex-4-ene-1,2-dicarboxylic anhydride, bicyclo[2.2.1]hept-5-ene2,3-dicarboxylic anhydride, and x-methylbicyclo[2.2.1]hept-5-ene-2,2-dicarboxylic anhydride.

Examples of other modifying groups include C₁-C₈ alkyl esters or glycidyl ester derivatives of unsaturated carboxylic acids such as methyl acrylate, methyl methacrylate, ethyl acrylate, ethyl methacrylate, butyl acrylate, butyl methacrylate, glycidyl acrylate, glycidal methacrylate, monoethyl maleate, diethyl maleate, monomethyl maleate, diethyl maleate, monomethyl fumarate, dimethyl fumarate, monomethyl itaconate, and diethylitaconate; amide derivatives of unsaturated carboxylic acids such as acrylamide, methacrylamide, maleicmonoamide, maleic diamide, maleic N-monoethylamide, maleic N,N-dietylamide, maleic N-monobutylamide, maleic N,N dibutylamide, fumaric monoamide, fumaric diamide, fumaric N-monoethylamide, fumaric N,N-diethylamide, fumaric N-monobutylamide and fumaric N,N-dibutylamide; imide derivatives of unsaturated carboxylic acids such as maleimide, N-butymaleimide and N-phenylmaleimide; and metal salts of unsaturated carboxylic acids such as sodium acrylate, sodium methacrylate, potassium acrylate and potassium methacrylate. More preferably, the polyolefin is modified by a fused ring carboxylic anhydride and most preferably a maleic anhydride.

The polyolefins also include ethylene vinyl acetate copolymers, modified ethylene vinyl acetate copolymers and blends thereof. The modified EVA has an associated modifying group selected from the above listed modifying groups.

In one preferred form of the invention, the tubing 104 has a solution contact layer 110 of a modified EVA copolymer sold by BYNEL under the trade designation CXA, a core layer 108 of an EVOH and an outside layer 106 of a modified EVA. Such a film is symmetrical from a materials standpoint. According to a preferred form of the invention, such tubing will have layers of the following thickness ranges: outside layer 106 from about 0.002 inches to about 0.042 inches, the core layer 108 from about 0.016 inches to about 0.056 inches, the solution contact layer 110 of from about 0.002 inches to about 0.042 inches.

In another preferred form of the invention, the tubing 104 has a solution contact layer 110 of an EVOH, a core layer 108 of a modified EVA and preferably BYNEL CXA and an outside layer 106 of an ethylene and α-olefin copolymer. Such a film is symmetrical from a materials standpoint. The tubing layers can have various relative thicknesses. According to a preferred form of the invention, such tubing will have layers of the following thickness ranges: outside layer 106 from about 0.002 inches to about 0.042 inches, the core layer 108 from about 0.002 inches to about 0.042 inches, the solution contact layer 110 of from about 0.016 inches to about 0.056 inches.

In a preferred form of the invention, the closed end tube 26, 104 or 112 shall have the following dimensions: inside diameter from about 0.100 inches to about 0.500 inches and the wall thickness shall be from about 0.020 inches to about 0.064 inches.

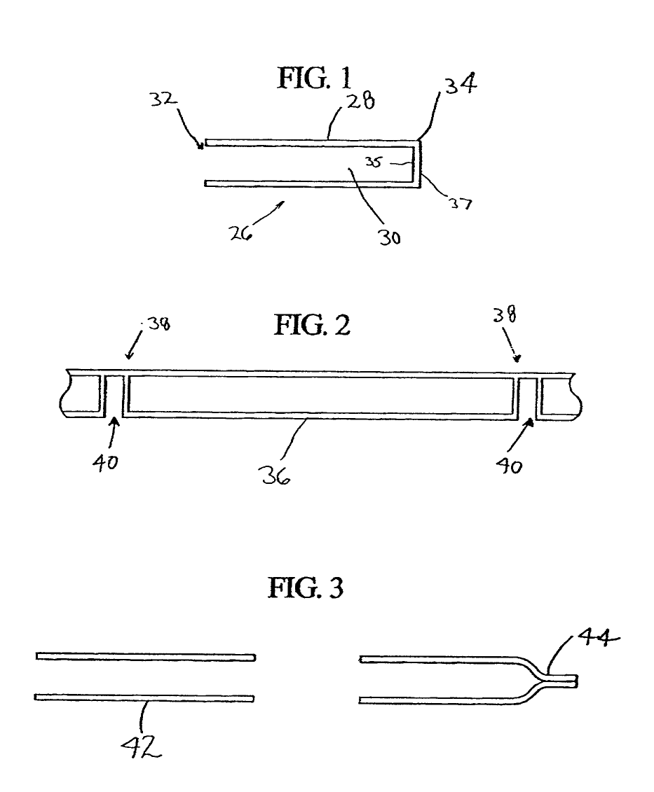

The closed end tube 26 may be made using any suitable process, but preferably is extrusion molded as shown in FIG. 2. FIG. 2 shows an extruded length 36. The length 36 repeats at spaced intervals along the length of the extrudate. At each end 38 of the length 36 is an interval 40 which defines the successive lengths 36. The lengths 36 are cut at the intervals 40 to leave a length 36 closed at each end 38. The length 36 is then cut again along its center, thereby giving two closed end tubes. Any excess material at the closed second ends of the lengths 36 remaining after cutting may be trimmed using any suitable means. Alternatively, the closed end tube 26 may be extruded as an open end tube 42 as shown in FIG. 3, and then one end 44 sealed to provide a closed end tube 26.

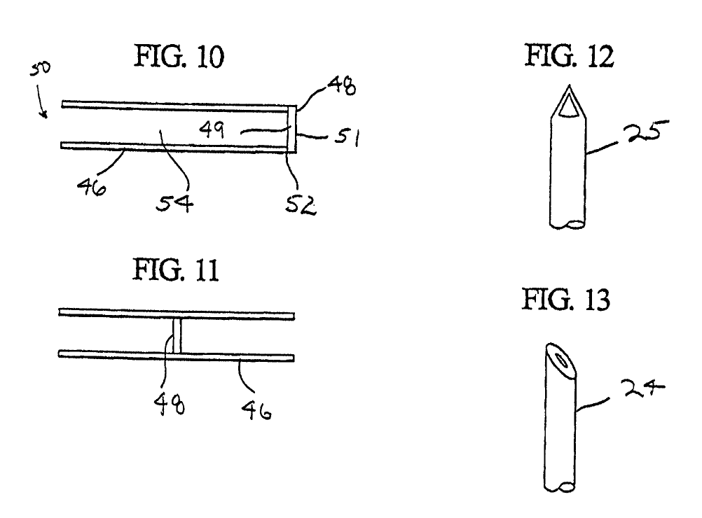

In another preferred form of the invention, membrane tube 19 inserted into the access port 18 to the container 12 may be an open end tube 46. A membrane 48 is attached to the open end tube 46 as shown in FIG. 10. The membrane 48 has an inner surface 49 and an outer surface 51. The open end tube 46 has a first end 50 in communication with the container 12, and a second end 52. A passageway 54 is defined by the first end 50 and second end 52. The membrane 48 is attached to the second end 52 of the open tube 46. The membrane 48 may be attached to the second end 52 using any suitable process, but preferably is attached by radio frequency welding. Alternatively, the membrane 48 may be disposed at a suitable point along the passageway 54 as in FIG. 11.

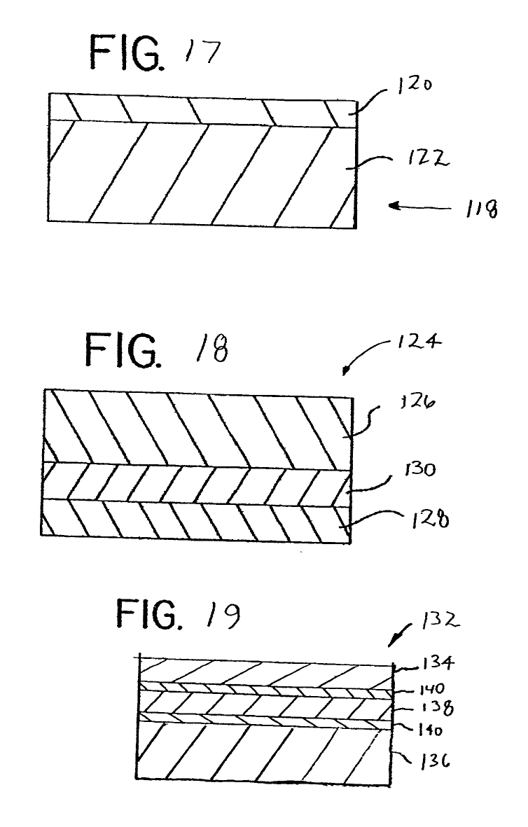

The open tube 46 is preferably made in the manner described above with respect to the closed end tube 26. The membrane 48 can have any number of layers, but in a preferred form of the invention has multiple layers. FIG. 17 shows a two-layered structure 118 having an outside layer 120 and an inside layer 122. FIG. 18 shows a three-layered structure 124 having an outside layer 126, an inside layer 128 and a core layer 130. FIG. 19 shows a five layered structure 132 having an outside layer 134, an inside layer 136, a core layer 138 and two tie layers 140. In a preferred form of the invention one layer shall be of a barrier material defined above and the remaining layer or layers shall be selected from the polyolefins defined above, polyamides and polyesters. One of the inside layers or outside layer shall define a tubing contact layer or seal layer.

Suitable polyamides include those obtained from a ring-opening reaction of lactams having from 4-12 carbons. This group of polyamides therefore includes, but is not limited to, nylon 6, nylon 10 and nylon 12.

Acceptable polyamides also include aliphatic polyamides resulting from the condensation reaction of di-amines having a carbon number within a range of 2-13, aliphatic polyamides resulting from a condensation reaction of di-acids having a carbon number within a range of 2-13, polyamides resulting from the condensation reaction of dimer fatty acids, and amide containing copolymers. Thus, suitable aliphatic polyamides include, for example, nylon 66, nylon 6,10 and dimer fatty acid polyamides.

Suitable polyesters include polycondensation products of di- or polycarboxylic acids and di or poly hydroxy alcohols or alkylene oxides. Preferably, the polyesters are a condensation product of ethylene glycol and a saturated carboxylic acid such as ortho or isophthalic acids and adipic acid. More preferably the polyesters include polyethyleneterphthalates produced by condensation of ethylene glycol and terephthalic acid; polybutyleneterephthalates produced by a condensations of 1,4-butanediol and terephthalic acid; and polyethyleneterephthalate copolymers and polybutyleneterephthalate copolymers which have a third component of an acid component such as phthalic acid, isophthalic acid, sebacic acid, adipic acid, azelaic acid, glutaric acid, succinic acid, oxalic acid, etc.; and a diol component such as 1,4-cyclohexanedimethanol, diethyleneglycol, propyleneglycol, etc. and blended mixtures thereof.

In a preferred form of the invention having a barrier layer, the membrane structure shall have five layers as shown in FIG. 19 and is a variation of the film structure disclosed in commonly assigned U.S. Pat. No. 6,083,587 which is incorporated herein by reference and made a part hereof. The outside layer 134 is a polyamide and preferably nylon 12, the two tie layers 140 are a modified EVA copolymer, the core layer 138 is an EVOH and the inner layer 136 is a modified EVA. In a preferred form of the invention the inside layer 136 defines the tubing contact layer.

Further, the structure shown in FIG. 19 shall have the following layer thickness ranges: outside layer 134 from about 0.0005 inches to about 0.003 inches, the tie layers 140 from about 0.0005 inches to about 0.02 inches, the core layer 138 of from about 0.0005 inches to about 0.0015 inches and an inside layer 136 of from about 0.008 inches to about 0.012 inches.

For membranes not using a barrier, the preferred membrane structure is a monolayer structure. The monolayer structure preferably comprises polypropylene and styrene ethylene butene styrene (SEBS), or kraton. The polypropylene and SEBS are preferably blended using 20-50% SEBS, and 50-80% polypropylene. Most preferably, the blend is about 30% SEBS and 70% polypropylene.

Another preferred membrane monolayer structure includes a MARQ material. The MARQ material includes about 10% SEBS, 45% polypropylene, 35% ultra low density polyethylene (ULDPE), and 10% di-mer fatty acid polyamide as disclosed in U.S. Pat. No. 5,849,843, fully incorporated as though made a part hereof. In a further preferred embodiment, the membrane monolayer structres is made entirely of ULDPE.

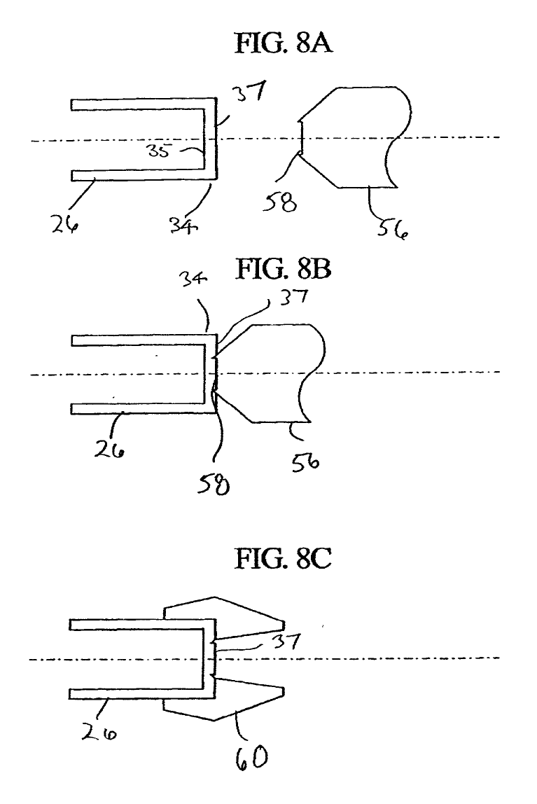

FIGS. 8A through 8C show a preferred method by which the closed second end 34 of the closed end tube 26 is contoured with a selected pattern to define a zone of weakness in the closed second end 34 in accord with the present invention. The preferred method contemplates heating by radio frequency, ultrasonic or thermal conduction to form the contouring. The contouring may also be formed by injection molding the contour pattern, by cold coining, by coining while injection molding using core pins, laser etching, solvent etching, machine cutting using a spinning tool, forging or stamping methods, or any suitable method.

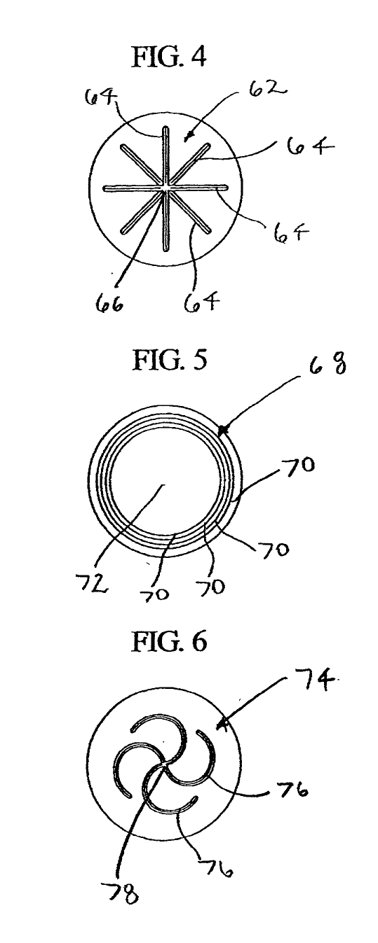

FIG. 8A illustrates the closed end tube 26 and a contour forming head 56. The contour forming head 56 contains at its working end 58 the selected contouring pattern. The pattern may include those of the embodiments illustrated in FIGS. 4 through 7, discussed below. In FIG. 8A, the contour forming head 56 is presented to the closed end tube 26.

FIG. 8B shows the contour forming head 56 partially penetrating the outer surface 58 of the closed second end 34 of the closed end tube 26. The contour forming head 56 thus forms the selected pattern in the outer surface 37 of the closed second end 34.

It should be understood that although the method has been described with respect to contouring the outer surface 37 of the closed second end 34, the method may also be used to contour the inner surface 35 of the closed second end 34. Moreover, while the preferred method has been described with respect to the embodiment employing the closed end tube 26, it is contemplated that the same method can be used to contour the open end tube/membrane and renal application embodiments described herein. It is further contemplated that the closed end tube may also include a membrane disposed within it, or that the membrane tube may have more than one membrane.

FIG. 8C illustrates an elastomeric spike holder 60 overmolded onto the contoured closed end tube 26. The elastomeric spike holder 60 engages the access spike and assists in holding the access spike in the tube during fluid delivery. The spike holder 60 may be of any type suitable for holding the access spike in place but is preferably as discussed below.

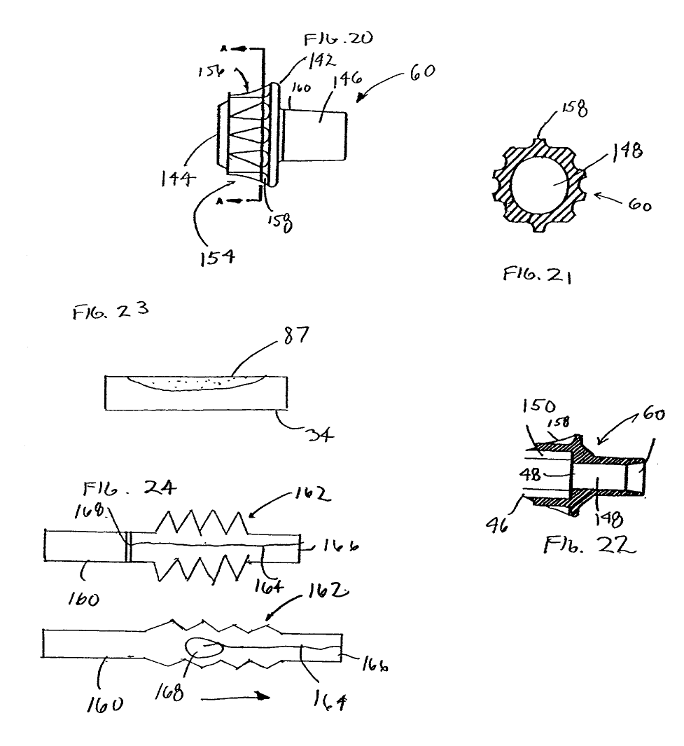

As shown in FIGS. 20-22, the spike holder 60 preferably has a body 142 having a first chamber 144 at a first end a second chamber 146 at a second end and a passageway 148 connecting the first and second chambers. FIGS. 20-22 illustrate the spike holder 60 in connection with the embodiment using a membrane 48 situated at the end of an open end tube 46 as described above. It will be understood that the spike holder 60 may be used with any embodiment described herein. The first chamber 144 is dimensioned to telescopically receive an end portion 150 of the tube 46. It is contemplated by the present invention the chamber 144 could extend into the tube fluid passageway 148 and attach thereto without departing from the present invention. The second chamber 146 is dimensioned to form an interference fit with an access spike 20. In a preferred form of the invention, the first chamber 144 and the second chamber 146 have a generally circular cross-sectional shape, the first chamber 144 having a first diameter and the second chamber 146 having a second diameter, the first diameter being larger than the second diameter.

In a preferred form of the invention, the spike holder 60 has an outwardly extending flange 154 at an intermediate portion thereof. The flange 14 is positioned generally at the intersection of the first chamber 144 and the second chamber 146. The flange 154 has a first surface 156 wherein a plurality of buttresses 158 extend from the first surface of the body 142. In a preferred form of the invention, the flange 154 is generally circular in cross-sectional shape and the buttresses 158 are circumferentially spaced about the first surface 156. The buttresses 158 are shown having a generally tear-drop shape, however, could be of numerous different shapes without departing from the present invention. The buttresses 158 are provided to form a gripping surface for those handling the spike holder 60.

The spike holder 60 is formed from a polyolefin as defined above and more particularly is an ethylene and α-olefin copolymer. The spike holder 60 can also have a textured or matte finish on a portion or the entire outer surface 160 of the holder 60 for ease of handling. The spike holder 60 can be formed by any suitable polymer forming technique known to those skilled in the art and preferably the spike holder 50 is formed by injection molding. The spike holder 60 can also include a membrane film positioned in the passageway 148 in lieu of or in addition to the membrane 48.

In a preferred form of the invention, the spike holder 60 is formed directly over the end portion 150 of the open end tube 46/membrane 48 assembly described above. Such a process shall be referred to as an overmolding process. The overmolding process includes the steps of: (1) providing a tubing as set forth above; providing a mold for forming a spike holder; inserting a portion of the tubing into the mold; and supplying polymeric material to the mold to form a spike holder on the tubing. While the preferred method has been described with respect to the embodiment employing the open end tube 46 and membrane 48, it is contemplated that the same method can be used to contour the closed end tube and renal application embodiments described herein.

FIG. 4 illustrates one preferred embodiment of the contouring pattern of the present invention. FIG. 4 shows a radial contouring pattern 62. The radial contouring pattern 62 preferably has a plurality of intersecting lines 64 that intersect at substantially the center point 66 of the closed second end 34 of the closed end tube 26, or the membrane 48. The radial contouring pattern 62 is preferably located on the outer surface 37 of the closed second end 34, but alternatively may be on the inner surface 35. The radial contouring pattern 62, due to the reduced thickness of the closed second end 34 along the pattern lines, creates a zone of weakness in the closed second end 34. The zone of weakness can either be discrete or not, and can extend beyond the pattern or be entirely within the pattern, or extend along the pattern. The zone of weakness permits reduced spike access force to the closed second end 34. The radial contouring pattern 62 favors use with a center point access spike 25.

FIG. 5 illustrates a concentric contouring pattern 68 representing another preferred embodiment of the present invention. The concentric contouring pattern 68 preferably includes a series of circles 70 arranged concentrically about a substantially center point 72 of the closed second end 34 of the closed end tube 26, but may be a single circle. The concentric contouring pattern 68 is preferably located on the outer surface 37 of the closed second end 34, but alternatively may be on the inner surface 35. The concentric contouring pattern 68, due to the reduced thickness of the closed second end 34 along the pattern lines, creates a zone of weakness in the closed second end 34. The zone of weakness can either be discrete or not, and can extend beyond the pattern or be entirely within the pattern, or extend along the pattern. The zone of weakness permits reduced spike access force to the closed second end 34. The concentric contouring pattern 68 favors use with a beveled access spike 24.

FIG. 6 illustrates a spiral contouring pattern 74 of a further preferred embodiment of the present invention. The spiral contouring pattern 74 preferably includes two intersecting spiral lines 76 that intersect a substantially the center point 78 of the closed second end 34 of the closed end tube 26, but may be a single spiral line. The spiral contouring pattern 74 is preferably located on the outer surface 37 of the closed second end 34, but alternatively may be on the inner surface 35. The spiral contouring pattern 74 combines the features of the radial and concentric contouring patterns of FIGS. 4 and 5. Due to the reduced thickness of the closed second end 34 along the pattern lines, a zone of weakness is created in the closed second end 34. The zone of weakness permits reduced spike access force to the closed second end 34. The zone of weakness can either be discrete or not, and can extend beyond the pattern or be entirely within the pattern, or extend along the pattern. The spiral contouring pattern 74 may be used with either a center point access spike 25 or beveled access spike 24.

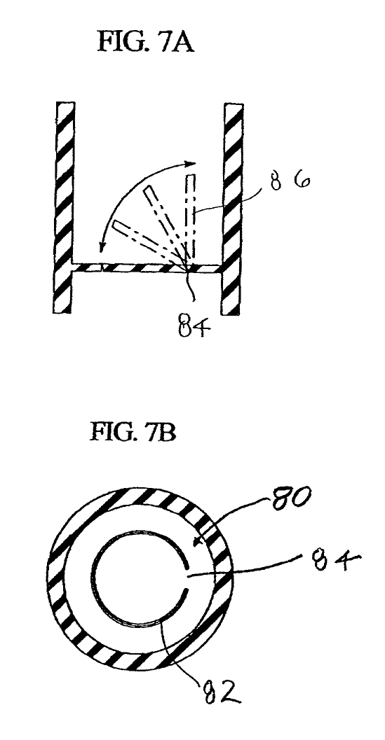

FIGS. 7A and 7B illustrate a hinged valve contouring pattern 80 of a further embodiment of the present invention. The hinged valve contouring pattern 80 of FIG. 7B has a circular contoured portion 82 and a hinged section 84. The circular portion 82 is preferably centrally located on the closed second end 34 of the closed end tube 26. The hinged valve contouring pattern 80, due to the reduced thickness of the closed second end 34 along the pattern lines, creates a zone of weakness in the closed second end 34. The zone of weakness permits reduced spike access force to the closed second end 34. The zone of weakness can either be discrete or not, and can extend beyond the pattern or be entirely within the pattern, or extend along the pattern.

When an access spike punctures the closed second end 34, the closed second end 34 breaks along the circular contoured portion 82 forming a flap 86. (FIG. 7A) The hinged section 84 rotates the flap 86 about the hinged section 84. When the access spike is removed, elastomeric properties of the hinged section 84 rotate the flap 86 back to its original position, thus resealing the closed second end of the closed second end 26, and inhibiting flow of fluid from the container 12. The zone of weakness can extend beyond the pattern or be entirely within the pattern, or extend along the pattern.

Additional patterns are also contemplated that include combinations of the above patterns, such as use of radial lines of FIG. 4 with concentric circles of FIG. 5. The pattern could also include a series of spaced perforations on the outer surface of the closed second end 26. For each of these patterns, the zone of weakness can extend beyond the pattern or be entirely within the pattern, or extend along the pattern. The contour lines that form the above patterns are preferably v-shaped in cross-section.

Also, it is contemplated that where multiple membranes are used, or where more than one membrane is also disposed within a closed end tube, the membranes and/or closed end of the closed end tube may have the same or differing patterns.

Moreover, as shown in FIG. 23, the closed end 34 of the closed end tube 26 may be weakened by using a solvent to created create a zone of weakness 87. Solvent weakening may be used by itself, or in conjunction with the patterns described above.

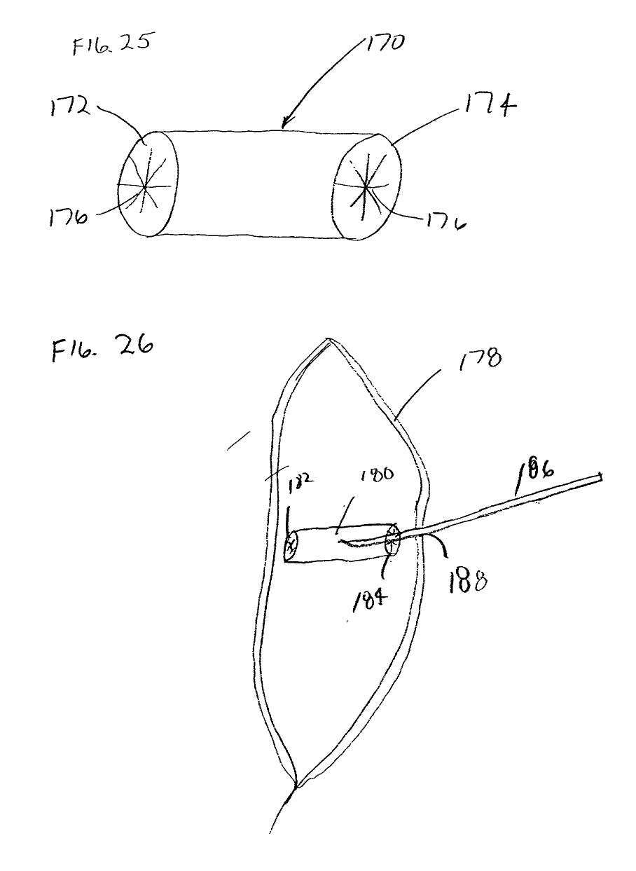

A further embodiment is shown in FIG. 24. A tube 160 includes a bellows 162. A tether 164 attached to the end 166 of the tube 160 extends through the tube 160 to a membrane 168 contoured or weakened as described above. When the bellows 162 is pulled in the direction of the arrow, the tether 164 pulls at least a portion of the membrane 168 from the sides of the tube 160 thereby permitting fluid flow. Alternatively, the tether 164 may extend out of the tube 160.

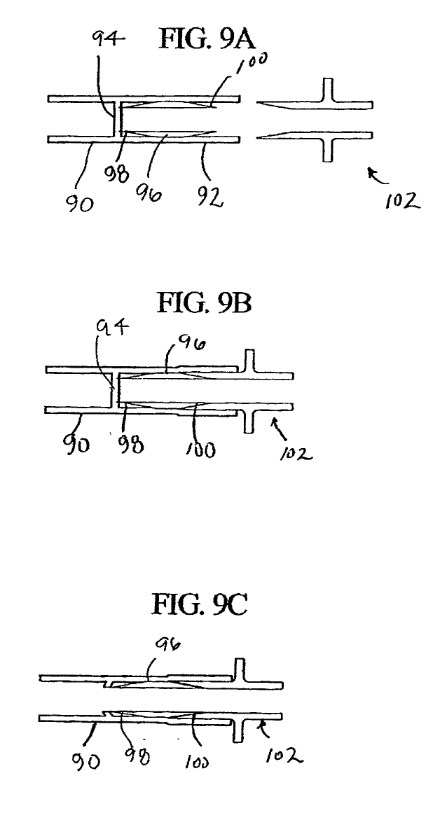

FIGS. 9A through 9C illustrate a further preferred embodiment of the present invention contemplated for use with inline frangibles for delivery of renal fluids. In this embodiment, a tube 90 having an end 92 contains a membrane 94 disposed across the tube 90. The membrane 94 is contoured with one of the patterns described above. A cannula interface 96 having a first end 98 and a second end 100 is placed in the tube 90 between the membrane 94 and the end 92.

A connector 102 is designed to engage with the second end 100 of the cannula interface 96 while in the tube 90 as shown in FIG. 9B. When the connector 102 is inserted into the tube 90, it engages the second end 100 of the cannula interface 96. As shown in FIG. 9C, further engagement of the connector 102 pushes the first end 98 of the cannula interface 96 through the contoured membrane 94 thereby enabling renal fluid delivery.

While the contouring pattern embodiments of FIGS. 4 through 7 have been described with respect to the embodiment employing the closed end tube 26, it is contemplated that the same method can be used to contour the membrane and membrane renal application embodiments described herein. For the membrane embodiment described herein, substitute end tube 46 and membrane 48 for the closed end tube 26 and second end 34, respectively. For the renal application embodiment, substitute tube 90 and membrane 94 for the closed end tube 26 and second end 34, respectively.

In a further embodiment of the present invention (FIG. 25), a capsule 170 has a first end 172 and a second end 174. The first end 172 and second end 174 preferably are contoured with a pattern 176 to define a zone of weakness as described above. The capsule 170 can be placed in a pouch or other squeezable container (not shown). For instance, the capsule 170 may contain soda syrup, and may be placed in a pouch containing carbonated water. By squeezing the pouch, the capsule 170 is also compress. The compression causes one or both of the first end 172 and second end 174 to fail at their zones of weakness. Upon shaking the pouch, the soda syrup inside the capsule 170 mixes with the carbonated water inside the pouch to create a soda drink.

In a still further embodiment (FIG. 26), a container 178 has within it a capsule 180 which may be constructed in accord with the embodiment of FIG. 25. The capsule 180 has a first end 182 and a second end 184 which preferably both contain patterns defining a zone of weakness as described above. A stick 186 is inserted into a leak-proof opening 188 in the container 178. The stick 186 may be a straw or other suitable device. The capsule 180 is oriented such that when the stick 186 is inserted into the container 178, it punctures at least on end 182 or 184 of the capsule 180. As in the embodiment of FIG. 25, the container 178 may contain carbonated water, and the capsule 180 may contain soda syrup such that when the capsule 180 is punctured, the syrup mixes with the carbonated water to make soda. In addition to soda and syrup, the embodiments of FIGS. 25 and 26 contemplate use with reconstituting drugs, for instance, the capsule 180 can contain a drug used to reconstitute that contained in container 178.

The invention is claimed as follows:

1. A method for delivering fluid comprising the steps of:
providing a container to hold the fluid;
providing a closed-end tube having a first end and a second end, the first end in communication with the container, and the second end being closed, the closed second end further having an inner surface and an outer surface; and
contouring the closed second end in a pattern to form a zone of weakness.

2. The method of claim 1 wherein the step of contouring comprises treating the outer surface of the closed second end.

3. The method of claim 1 wherein the step of contouring comprises treating the inner surface of the closed second end.

4. The method of claim 1 wherein the pattern comprises at least one line extending radially from a point substantially central to the closed second end.

5. The method of claim 1 wherein the pattern is substantially circular and is generally centrally disposed on the closed second end.

6. The method of claim 1 wherein the pattern comprises at least one line extending spirally from a point substantially central to the closed second end.

7. The method of claim 1 wherein the step of contouring comprises the step of treating the closed second end ultrasonically.

8. The method of claim 1 wherein the step of contouring comprises the step of treating the closed second end by radio frequency heating.

9. The method of claim 1 wherein the step of contouring comprises the step of treating the closed second end by coining.

10. The method of claim 1 wherein the pattern comprises at least one line substantially intersecting the center point of the closed second end.

11. The method of claim 1 wherein the pattern comprises at least one generally curved line substantially intersecting the center point of the closed second end.

12. The method of claim 1 wherein the pattern comprises at least one curved line.

13. The method of claim 1 wherein the pattern comprises at least one substantially straight line.

14. The method of claim 1 wherein the pattern comprises at least one circular line.

15. The method of claim 1 wherein the zone of weakness is centrally disposed on the closed second end.

16. The method of claim 1 wherein the zone of weakness is larger than the pattern.

17. The method of claim 1 wherein the zone of weakness is smaller than the pattern.

18. The method of claim 1 wherein the pattern defines a hinged flap connected to the closed second end.

19. The method of claim 1 wherein the zone of weakness defines a hinged flap connected to the closed second end.

20. A fluid delivery system comprising
a container to hold the fluid;
a closed-end tube having a first end and a second end, the first end in communication with the container, and the second end being closed, the closed second end further having an inner surface and an outer surface; and
wherein the closed second end is contoured in a pattern to form a zone of weakness.

21. The system of claim 20 wherein the pattern is located on the outer surface of the closed second end.

22. The system of claim 20 wherein the pattern is located on the inner surface of the closed second end.

23. The system of claim 20 wherein the pattern comprises at least one line extending radially from a point substantially central to the closed second end.

24. The system of claim 20 wherein the pattern is substantially circular and is generally centrally disposed on the closed second end.

25. The system of claim 20 wherein the pattern comprises at least one line extending spirally from a point substantially central to the closed second end.

26. The system of claim 20 wherein the pattern is applied to the closed second end ultrasonically.

27. The system of claim 20 wherein the pattern is applied to the closed second end by radio frequency heating.

28. The system of claim 20 wherein pattern is applied to the closed second end by coining.

29. The system of claim 20 wherein the pattern comprises at least one line substantially intersecting the center point of the closed second end.

30. The system of claim 20 wherein the pattern comprises at least one generally curved line substantially intersecting the center point of the closed second end.

31. The system of claim 20 wherein the pattern comprises at least one curved line.

32. The system of claim 20 wherein the pattern comprises at least one substantially straight line.

33. The system of claim 20 wherein the pattern comprises at least one circular line.

34. The system of claim 20 wherein the zone of weakness is centrally disposed on the closed second end.

35. The system of claim 20 wherein the zone of weakness is larger than the pattern.

36. The system of claim 20 wherein the z one of weakness is smaller than the pattern.

37. The system of claim 20 wherein the pattern defines a hinged flap connected to the closed second end.

38. The system of claim 20 wherein the zone of weakness defines a hinged flap connected to the closed second end.

39. A method for delivering fluid comprising the steps of:
providing a container for holding the fluid;
providing a passageway in communication with the container;
disposing a membrane across the passageway to seal the passageway, the membrane having a inner surface and an outer surface; and
contouring the membrane in a pattern to define a zone of weakness.

40. The method of claim 39 wherein the passageway has a first end in communication with the container and a second free end, and wherein the membrane is located at the second free end of the passageway.

41. The method of claim 39 wherein the passageway has a first end in communication with the container and a second free end, and wherein the membrane is located between the first and second ends of the passageway.

42. The method of claim 39 wherein the step of contouring comprises treating the outer surface of the closed second end.

43. The method of claim 39 wherein the step of contouring comprises treating the inner surface of the closed second end.

44. The method of claim 39 wherein the pattern comprises at least one line extending radially from a point substantially central to the closed second end.

45. The method of claim 39 wherein the pattern is substantially circular and is generally centrally disposed on the closed second end.

46. The method of claim 39 wherein the pattern comprises at least one line extending spirally from a point substantially central to the closed second end.

47. The method of claim 39 wherein the step of contouring comprises the step of treating the closed second end ultrasonically.

48. The method of claim 39 wherein the step of contouring comprises the step of treating the closed second end by radio frequency heating.

49. The method of claim 39 wherein the step of contouring comprises the step of treating the closed second end by coining.

50. The method of claim 39 wherein the pattern comprises at least one line substantially intersecting the center point of the closed second end.

51. The method of claim 39 wherein the pattern comprises at least one generally curved line substantially intersecting the center point of the closed second end.

52. The method of claim 39 wherein the pattern comprises at least one curved line.

53. The method of claim 39 wherein the pattern comprises at least one substantially straight line.

54. The method of claim 39 wherein the pattern comprises at least one circular line.

55. The method of claim 39 wherein the zone of weakness is centrally disposed on the closed second end.

56. The method of claim 39 wherein the zone of weakness is larger than the pattern.

57. The method of claim 39 wherein the zone of weakness is smaller than the pattern.

58. The method of claim 39 wherein the pattern defines a hinged flap connected to the closed second end.

59. The method of claim 39 wherein the zone of weakness defines a hinged flap connected to the closed second end.

60. A fluid delivery system comprising:
a container for holding the fluid;
a passageway in communication with the container;
a membrane disposed across the passageway to seal the passageway, the membrane having a inner surface and an outer surface; and
the membrane contoured in a pattern to define a zone of weakness.

61. The system of claim 60 wherein the passageway has a first end in communication with the container and a second free end, and wherein the membrane is located at the second free end of the passageway.

62. The system of claim 60 wherein the passageway has a first end in communication with the container and a second free end, and wherein the membrane is located between the first and second ends of the passageway.

63. The system of claim 60 wherein the pattern is located on the outer surface of the closed second end.

64. The system of claim 60 wherein the pattern is located on the inner surface of the closed second end.

65. The system of claim 60 wherein the pattern comprises at least one line extending radially from a point substantially central to the closed second end.

66. The system of claim 60 wherein the pattern is substantially circular and is generally centrally disposed on the closed second end.

67. The system of claim 60 wherein the pattern comprises at least one line extending spirally from a point substantially central to the closed second end.

68. The system of claim 60 wherein the pattern is applied to the closed second end ultrasonically.

69. The system of claim 60 wherein the pattern is applied to the closed second end by radio frequency heating.

70. The system of claim 60 wherein pattern is applied to the closed second end by coining.

71. The system of claim 60 wherein the pattern comprises at least one line substantially intersecting the center point of the closed second end.

72. The system of claim 60 wherein the pattern comprises at least one generally curved line substantially intersecting the center point of the closed second end.

73. The system of claim 60 wherein the pattern comprises at least one curved line.

74. The system of claim 60 wherein the pattern comprises at least one substantially straight line.

75. The system of claim 60 wherein the pattern comprises at least one circular line.

76. The system of claim 60 wherein the zone of weakness is centrally disposed on the closed second end.

77. The system of claim 60 wherein the zone of weakness is larger than the pattern.

78. The system of claim 60 wherein the zone of weakness is smaller than the pattern.

79. The system of claim 60 wherein the pattern defines a hinged flap connected to the closed second end.

80. The system of claim 60 wherein the zone of weakness defines a hinged flap connected to the closed second end.

81. A method for delivering fluid comprising the steps of:
providing a container for holding the fluids;
providing a tube having a first end and a second end and defining a passageway in communication with the container, the tube having a membrane disposed across the passageway between the first and second ends;
contouring the membrane in a pattern to define a zone of weakness;
placing an interface in the tube between the membrane and the second end of the tube;
inserting a connector into the second end of the tube, the connector adapted to engage the interface; and
the interface puncturing the membrane thereby delivering the fluid through the passageway.

82. The method of claim 81 wherein the step of contouring comprises treating the outer surface of the membrane.

83. The method of claim 81 wherein the step of contouring comprises treating the inner surface of the membrane.

84. The method of claim 81 wherein the pattern comprises at least one line extending radially from a point substantially central to the membrane.

85. The method of claim 81 wherein the pattern is substantially circular and is generally centrally disposed on the membrane.

86. The method of claim 81 wherein the pattern comprises at least one line extending spirally from a point substantially central to the membrane.

87. The method of claim 81 wherein the step of contouring comprises the step of treating the membrane ultrasonically.

88. The method of claim 81 wherein the step of contouring comprises the step of treating the membrane by radio frequency heating.

89. The method of claim 81 wherein the step of contouring comprises the step of treating the membrane by coining.

90. The method of claim 81 wherein the pattern comprises at least one line substantially intersecting the center point of the membrane.

91. The method of claim 81 wherein the pattern comprises at least one generally curved line substantially intersecting the center point of the membrane.

92. The method of claim 81 wherein the pattern comprises at least one curved line.

93. The method of claim 81 wherein the pattern comprises at least one substantially straight line.

94. The method of claim 81 wherein the pattern comprises at least one circular line.

95. The method of claim 81 wherein the zone of weakness is centrally disposed on the membrane.

96. The method of claim 81 wherein the zone of weakness is larger than the pattern.

97. The method of claim 81 wherein the zone of weakness is smaller than the pattern.

98. The method of claim 81 wherein the pattern defines a hinged flap connected to the membrane.

99. The method of claim 81 wherein the zone of weakness defines a hinged flap connected to the membrane.

100. A system for delivering fluid comprising:
a container for holding the fluid;
a tube having first and second ends and defining a passageway in communication with the container, the tube having a membrane disposed across the passageway between the first and second ends, the membrane contoured in a pattern to define a zone of weakness;
an interface in the tube between the membrane and the second end of the tube;
a connector adapted to be inserted into the second end of the tube, the connector also adapted to engage the interface, and to cause the interface to puncture the membrane thereby delivering the fluid through the passageway.

101. The system of claim 100 wherein the pattern is located on the outer surface of the membrane.

102. The system of claim 100 wherein the pattern is located on the inner surface of the membrane.

103. The system of claim 100 wherein the pattern comprises at least one line extending radially from a point substantially central to the membrane.

104. The system of claim 100 wherein the pattern is substantially circular and is generally centrally disposed on the membrane.

105. The system of claim 100 wherein the pattern comprises at least one line extending spirally from a point substantially central to the membrane.

106. The system of claim 100 wherein the pattern is applied to the membrane ultrasonically.

107. The system of claim 100 wherein the pattern is applied to the membrane by radio frequency heating.

108. The system of claim 100 wherein the pattern is applied to the membrane by coining.

109. The system of claim 100 wherein the pattern comprises at least one line substantially intersecting the center point of the membrane.

110. The system of claim 100 wherein the pattern comprises at least one generally curved line substantially intersecting the center point of the membrane.

111. The system of claim 100 wherein the pattern comprises at least one curved line.

112. The system of claim 100 wherein the pattern comprises at least one substantially straight line.

113. The system of claim 100 wherein the pattern comprises at least one circular line.

114. The system of claim 100 wherein the zone of weakness is centrally disposed on the membrane.

115. The system of claim 100 wherein the zone of weakness is larger than the pattern.

116. The system of claim 100 wherein the zone of weakness is smaller than the pattern.

117. The system of claim 100 wherein the pattern defines a hinged flap connected to the membrane.

118. The system of claim 100 wherein the zone of weakness defines a hinged flap connected to the membrane.

119. A capsule comprising:
a body;
the body having a first end and a second end, and at least one of the first end or second end contoured to define a zone of weakness.

120. A fluid mixing system comprising:
a capsule;
the capsule having a first end and a second end, and at least one of the first end or second end contoured to define a zone of weakness;
the capsule contained within a container, the capsule containing a first material, and the container containing a second material.
